(19) United States
(12) Patent Application Publication
(10) Pub. No.: US 2010/0144773 A1
(43) Pub. Date: Jun. 10, 2010

(54) PHARMACEUTICAL COMPOSITIONS FOR INTRANASAL ADMINISTRATION COMPRISING CHOLINE SALTS OF SUCCINIC ACID

(76) Inventor: Igor Anatolievich Pomytkin, Moscow (RU)

Correspondence Address: NOTARO, MICHALOS & ZACCARIA P.C., 100 DUTCH HILL ROAD, ORANGEBURG, NY, 10962, US

(21) Appl. No.: 12/670,696

(22) PCT Filed: Aug. 2, 2007
(86) PCT No.: PCT/RU07/00420

§ 371 Date: Feb. 3, 2010

(51) Int. Cl. A61K 31/205; A61P 25/28; A61P 25/16; A61P 9/10; A61K 31/51

(52) U.S. Cl. 514/276; 514/554

(57) ABSTRACT

The present invention relates to pharmaceutical compositions for the intranasal administration comprising a therapeutically effective amount of a compound of formula (I) or pharmaceutically acceptable salts thereof. Preferably, the pharmaceutically acceptable salt is selected from the group consisting of choline salt, sodium salt, potassium salt, and thiamine salt. Further, the present invention relates to methods for intranasal delivering compound of formula (I) and pharmaceutically acceptable salts thereof. Further, the present invention relates to methods for treating a neurodegenerative disease with using compositions of the present invention.

### FIELD OF THE INVENTION

The invention relates to the use of choline salts of succinic acid in pharmaceutical compositions for intranasal administration, particularly in compositions with a neuroprotective activity.

### BACKGROUND OF THE INVENTION

Choline salts of succinic acid are biologically active substances.

RU patent 2228147 discloses the use of dicholine salt of succinic acid for the treatment dyslipidemia, hyperlipidemia, and insulin resistance.

RU patent 2281766 discloses the method for improving cognitive function with the use of dicholine salt of succinic acid.

RU patent 2281765 discloses the method for the treatment of cerebral ischemia with the use of dicholine salt of succinic acid.

Monocholine salt of succinic acid is known from the prior art and is disclosed, for example, by published U.S. Pat. No. 5,124,061 as a component of compositions for increasing the resistance of plants to damage by freezing conditions.

However, there are no data in the art on the use of monocholine or dicholine salts of succinic acid as components in pharmaceutical compositions suitable for intranasal administration.

Surprisingly, it is demonstrated in the present invention that choline salts of succinic acid manifest a pronounced effect on brain function under intranasal administration, and these effects are achieved with doses tens and hundreds times less than required to achieve the same or even less effect when these salts are administered intraperitoneally. Moreover, intranasal route of administration of a choline salt of succinic acid provides desirable central effects and avoids undesirable systemic effects such as undesirable insulin secretion in response to injections of choline salts of succinic acids. Thus, because of intranasal administration of choline salts of succinic acid is now possible (1) to decrease effective doses of these salts and nevertheless to achieve desired central therapeutic effects and (2) to avoid undesired systemic adverse effects typical for other routes of delivery.

It is an object of the present invention to provide a pharmaceutical composition for the intranasal administration comprising a therapeutically effective amount of choline salts of succinic acid.

It is an object of the present invention to provide a method for treating neurodegenerative diseases comprising intranasally administering a pharmaceutical composition comprising a therapeutically effective amount of choline salts of succinic acid.

### DETAILED DESCRIPTION OF THE INVENTION

The present invention provides a pharmaceutical composition for the intranasal administration comprising a therapeutically effective amount of a compound of Formula (I)

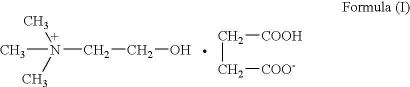

or a pharmaceutically acceptable salt thereof, and a pharmaceutically and intranasally acceptable carrier.

The term <<pharmaceutically acceptable salt” refers to non-toxic base addition salts. The pharmaceutically acceptable salts of the invention are prepared by a reaction of compound of formula (I) with a pharmaceutically acceptable base by methods well-known from the art. Such bases include, but are not limited to, ammonia; sodium base; potassium base; choline base; thiamine base; organic amines like as triethylamine, ethanolamine, dimethylethanolamine, diethanolamine, and triethanolamine; 2-ethyl-6-methyl-3-hydroxypiridine; and basic amino acids like arginine, ornithine, and lysine. Preferably, the pharmaceutically acceptable salt of the invention is selected from the group consisting of choline salt (compound of formula II), sodium salt (compound of formula III), potassium salt (compound of formula IV), and thiamine salt (compound of formula V).

The present invention further provides a pharmaceutical composition for the intranasal administration comprising a therapeutically effective amount of a compound of Formula (II)

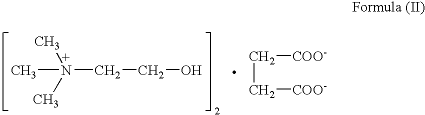

and a pharmaceutically and intranasally acceptable carrier.

The present invention further provides a pharmaceutical composition for the intranasal administration comprising a therapeutically effective amount of a compound of Formula (III)

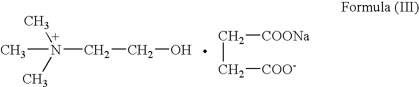

and a pharmaceutically and intranasally acceptable carrier.

The present invention further provides a pharmaceutical composition for the intranasal administration comprising a therapeutically effective amount of a compound of Formula (IV)

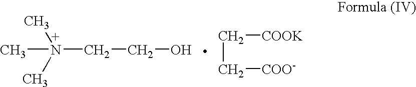

and a pharmaceutically and intranasally acceptable carrier.

The present invention further provides a pharmaceutical composition for the intranasal administration comprising a therapeutically effective amount of a compound of Formula (V)

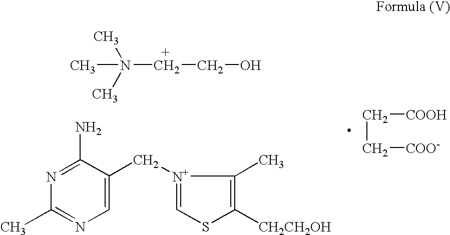

and a pharmaceutically and intranasally acceptable carrier.

The term “therapeutically effective amount” refers to a nontoxic but sufficient amount of an active agent to provide the desired therapeutic effect. Preferably, the therapeutically effective amount of compounds of formula (I) through (V) is from 0.01 to 30 mg per a unit dosage form of compositions of the present invention. More preferably, from 5 to 15 mg per a unit dosage form.

The term “intranasal administration” refers to delivery of the composition to any portion of the nasal epithelium.

The term <<pharmaceutically and intranasally acceptable carrier” refers to a one or more compatible solid or liquid filler diluents or encapsulating substances which are suitable for administration to any portion of the nasal epithelium of a mammal, preferably a human. Typically, the carrier may be a liquid, solution, suspension, gel, ointment, lotion, or combinations thereof. Preferably, the carrier is a pharmaceutically acceptable aqueous carrier.

The compositions of the invention are prepared by methods well-known from the art in accordance with accepted pharmaceutical procedures, for example, as described in Remington's Pharmaceutical Sciences, seventeenth edition, ed. Alfonso R. Gennaro, Mack Publishing Company, Easton, Pa., Eighteenth edition (1990).

The compositions of the invention can be prepared in a variety of unit dosage forms. Such forms are include, but are not limited to, nasal drop, nasal spray, nasal gel, nasal ointment, and nasal powder. The content of compounds of formula (I) through (V) is in the range from 0.1 to 99%, preferably 0.5 to 10% by the weight of the composition.

The present invention further provides a method for delivering a compound of Formula (I)

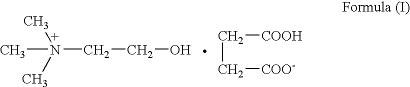

to a mammal in need thereof comprising intranasally administering a pharmaceutical composition comprising a therapeutically effective amount of the compound of Formula (I) or a pharmaceutically acceptable salt thereof, and a pharmaceutically and intranasally acceptable carrier. Preferably, the pharmaceutically acceptable salt is selected from the group consisting of choline salt, sodium salt, potassium salt, and thiamine salt.

The present invention further provides a method of treating a disease selected from the group consisting of Alzheimer's disease, Parkinson's disease, Huntington's disease, cerebral ischemia and neurological damage due to stroke, diabetic polyneuropathy, and amyotrophic lateral sclerosis; the method comprising intranasally administering to a mammal in need thereof a pharmaceutical composition comprising a therapeutically effective amount of the compound of Formula (I)

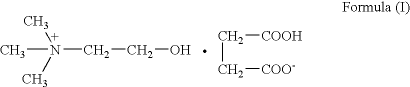

or a pharmaceutically acceptable salt thereof, and a pharmaceutically and intranasally acceptable carrier. Preferably, the pharmaceutically acceptable salt is selected from the group consisting of choline salt, sodium salt, potassium salt, and thiamine salt.

As used herein, the term “treating a disease” means treating, controlling, preventing and/or reducing one or more clinical signs (i.e., symptoms) of the disease in a mammal in need thereof.

Preferably, the therapeutically effective amount in the method of the present invention is 0.01 to 5 mg per kilogram of body weight of the mammal, to more preferably, 0.1 to 1 mg per kilogram.

Nonexclusive examples of mammals of the invention include humans and companion animals such as cats and dogs. Preferably, the mammal is a human.

The following examples are presented to demonstrate the invention. The examples are illustrative only and are not intended to limit the scope of the invention in any way.

### Example 1

This example demonstrates preparation of compounds of formula (I) through (V).

A compound of formula (I) is prepared by mixing 12.1 g choline base with 11.8 g succinic acid at room temperature without of a solvent. Resulting mixture is dissolved in acetone at ambient temperature; and the solution is filtered through a filter. Compound (I) is recovered as ionic liquid by evaporating of acetone from the solution. ¹H NMR in D₂O: 2.41 (9H, s), 3.19 (4H, s), 3.49 (2H, t), 4.10 (2H, t). Formula: C9H19NO5. Found: C 48.82%, H 8.69%, and N 6.30%. Calculated: C 48.86%, H 8.66%, and N 6.33%.

A compound of formula (II) is prepared by mixing of 2.2 g of the compound of formula (I) with 1.2 g of choline base at ambient temperature without of a solvent. The mixture is dried under vacuum and re-crystallized from isopropanol-acetone. Compound (II) is recovered as a white powder. ¹H NMR in D₂O: 2.37 (18H, s), 3.14 (4H, s), 3.49 (4H, t), 4.05 (4H, t). Formula: C14H32N2O6. Found: C 51.79%, H 9.98%, and N 8.60%. Calculated: C 51.83%, H 9.94%, and N 8.63%.

A compound of formula (III) is prepared by mixing of 2.2 g of the compound of formula (I) with 0.04 g of sodium hydroxide at ambient temperature without of a solvent. The mixture is dried under vacuum and re-crystallized from isopropanol-acetone. Compound (III) is recovered as a white powder. ¹H NMR in D₂O: 2.35 (9H, s), 3.15 (4H, s), 3.46 (2H, t), 4.00 (2H, t). Formula: C9H18NO5Na. Found: C 44.40%, H 7.49%, and N 5.72%. Calculated: C 44.44%, H 7.46%, and N 5.76%.

A compound of formula (IV) is prepared by mixing of 2.2 g of the compound of formula (I) with 0.056 g of potassium hydroxide at ambient temperature without of a solvent. The mixture is dried under vacuum and re-crystallized from isopropanol-acetone. Compound (IV) is recovered as a white powder. ¹H NMR in D₂O: 2.31 (9H, s), 3.10 (4H, s), 3.45 (2H, t), 4.02 (2H, t). Formula: C9H18NO5K. Found: C 41.63%, H 7.02%, and N 5.34%. Calculated: C 41.68%, H 6.99%, and N 5.40%.

A compound of formula (V) is prepared by mixing of 2.2 g of the compound of formula (I) with 2.82 g of thiamine base at ambient temperature without of a solvent. The mixture is dried under vacuum and re-crystallized from isopropanol-acetone. Compound (V) is recovered as a white powder. Formula: C21H35N5O6S. Found: C 51.90%, H 7.31%, and N 14.39%. Calculated: C 51.94%, H 7.27%, and N 14.42%.

### Example 2

This example demonstrates compositions for intranasal administration comprising compound of formula (I).

| Ingredient | Content |
|---|---|
| Compound of formula (I) | 50 mg/ml |
| Disodium phosphate USP/Ph Eur | qs to pH 5.0 |
| Water for injections USP/Ph Eur | to 1.0 ml |

Compound of formula (I) is dissolved in water for injection to the desired volume, 0.4M disodium phosphate is added to pH 5.0. In this manner, solution with concentration of compound of formula (I) of 50 mg/ml is prepared. The solution is filtered through a sterilizing grade filter (0.2 μm), and filled into USP/Ph Eur Type 1 glass vials (nominal spray volume 100 μL) which are closed with chlorobutyl stoppers. The vials are assembled into the commercially available unit dose nasal spray device. The assembled device may be used to deliver unit doses of compound of formula (I) of 5.0 mg in a single administration. The filled nasal spray device is packaged into a plastic tray and placed inside a carton to provide protection from the light.

### Example 3

This example demonstrates compositions for intranasal administration comprising compound of formula (II).

| Ingredient | Content |
|---|---|
| Compound of formula (II) | 50 mg/ml |
| Succinic acid | qs to pH 5.0 |
| Water for injections USP/Ph Eur | to 1.0 ml |

Compound of formula (II) is dissolved in water for injection to the desired volume, 0.4M succinic acid is added to pH 5.0. In this manner, solution with concentration of compound of formula (II) of 50 mg/ml is prepared. The solution is sterilized and filled into glass vials as described in Example 1. The vials are assembled into the commercially available unit dose nasal spray device. The assembled device may be used to deliver unit doses of compound of formula (II) of 5.0 mg in a single administration. The filled nasal spray device is packaged into a plastic tray and placed inside a carton to provide protection from the light.

### Example 4

This example demonstrates compositions for intranasal administration comprising compound of formula (III).

| Ingredient | Content |
|---|---|
| Compound of formula (III) | 50 mg/ml |
| Succinic acid | qs to pH 5.0 |
| Water for injections USP/Ph Eur | to 1.0 ml |

Compound of formula (III) is dissolved in water for injection to the desired volume, 0.4M succinic acid is added to pH 5.0. In this manner, solution with concentration of compound of formula (III) of 50 mg/ml is prepared. The solution is sterilized and filled into glass vials as described in Example 1. The vials are assembled into the commercially available unit dose nasal spray device. The assembled device may be used to deliver unit doses of compound of formula (III) of 5.0 mg in a single administration. The filled nasal spray device is packaged into a plastic tray and placed inside a carton to provide protection from the light.

### Example 5

This example demonstrates compositions for intranasal administration comprising compound of formula (IV).

| Ingredient | Content |
|---|---|
| Compound of formula (IV) | 50 mg/ml |
| Succinic acid | qs to pH 5.0 |
| Water for injections USP/Ph Eur | to 1.0 ml |

Compound of formula (IV) is dissolved in water for injection to the desired volume, 0.4M succinic acid is added to pH 5.0. In this manner, solution with concentration of compound of formula (IV) of 50 mg/ml is prepared. The solution is sterilized and filled into glass vials as described in Example 1. The vials are assembled into the commercially available unit dose nasal spray device. The assembled device may be used to deliver unit doses of compound of formula (IV) of 5.0 mg in a single administration. The filled nasal spray device is packaged into a plastic tray and placed inside a carton to provide protection from the light.

### Example 6

This example demonstrates compositions for intranasal administration comprising compound of formula (V).

| Ingredient | Content |
|---|---|
| Compound of formula (V) | 50 mg/ml |
| Succinic acid | qs to pH 5.0 |
| Water for injections USP/Ph Eur | to 1.0 ml |

sCompound of formula (V) is dissolved in water for injection to the desired volume, 0.4M succinic acid is added to pH 5.0. In this manner, solution with concentration of compound of formula (V) of 50 mg/ml is prepared. The solution is sterilized and filled into glass vials as described in Example 1. The vials are assembled into the commercially available unit dose nasal spray device. The assembled device may be used to deliver unit doses of compound of formula (V) of 5.0 mg in a single administration. The filled nasal spray device is packaged into a plastic tray and placed inside a carton to provide protection from the light.

### Example 7

This example demonstrates methods for intranasal administration of compositions comprising compounds of formula (I) through (V).

The patient removes the packaging from the nasal spray device which contains a sterile solution of one of compounds of formula (I), (II), (III), (IV), or (V) and then inserts the nozzle of the device into a nostril and articles is to administer a single dose.

### Example 8

This example demonstrates methods for treating neurodegenerative disorders in mammals in need thereof.

A disease relevant to human Alzheimer's disease was induced by injection of beta-amyloid peptide 25-35 (beta-amyloid) into nucleus basalis magnocellularis (NBM) of rat brains as described by Harkany T et al. in Behav Brain Res. 1998 90(2):133-45. Beta-amyloid was administered bilaterally into NBM of male Wistar rats in dose of 2 μg per each side. On day 16^{th} after the amyloid injection, rats received intranasally or intraperitoneally compositions comprising a water solution of 1 mg/kg of compounds of formula (I), (II), (III), (IV), or (V) for 7 days singly a day. Control rats received saline intranasally. On day next to the last day of the treatment, passive avoidance performance in rats was tested for two consecutive days. A two-compartment, step-through, passive avoidance apparatus consisting of illuminated (25×40×25 cm) and dark (25×40×25 cm) compartments attached to an electrified grid floor and separated by a guillotine door (8×8 cm) was used. In the acquisition trial, the rat was placed in the illuminated compartment in a position its tail directed to the closed door for 2 min to habituate to the apparatus. The guillotine door was opened and time to enter to dark compartment was recorded. When the rat entered to dark compartment completely (four foots in dark compartment), the guillotine door was closed and the rat was delivered an electrical shock of 0.8 mA for 3 sec through the grid floor. After the shock, the rat was immediately placed in home cage. In the retention trial, conducted 24 h after the acquisition trial, the rat was placed in the illuminated compartment and the retention latency to enter into the dark compartment was recorded until 180 s had elapsed. The latency was accepted for 180 s, if the rat did not enter the dark compartment for 180 s. Data are presented as retention latency mean±SD (n=8).

| Groups | Latency, s |
|---|---|
| Control | 48 ± 11 |
| Compound of formula (I), intranasally | 103 ± 18* |
| Compound of formula (I), | 55 ± 18 |
| intraperitoneally | |
| Compound of formula (II), intranasally | 98 ± 21* |
| Compound of formula (II), | 45 ± 16 |
| intraperitoneally | |
| Compound of formula (III), | 87 ± 19* |
| intranasally | |
| Compound of formula (III), | 53 ± 28 |
| intraperitoneally | |
| Compound of formula (IV), | 65 ± 16* |
| intranasally | |
| Compound of formula (IV), | 49 ± 17 |
| intraperitoneally | |
| Compound of formula (V), intranasally | 92 ± 17* |
| Compound of formula (V), | 59 ± 23 |
| intraperitoneally | |

*Differs significantly of control (P < 0.05).

Thus, intranasal administration of composition comprising compounds of formula (I) through (V) is much more effective than intraperitoneal administration. Intranasally treated rats demonstrate significant improvement in learning and memory as compared to control rats, whereas intraperitoneally treated rats do not.

1. A pharmaceutical composition for the intranasal administration comprising a therapeutically effective amount of a compound of Formula (I) 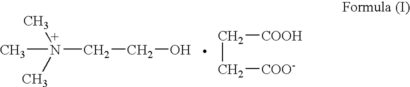
or a pharmaceutically acceptable salt thereof, and a pharmaceutically and intranasally acceptable carrier.

2. The composition of claim 1, wherein the pharmaceutically acceptable salt is a compound of Formula (II).

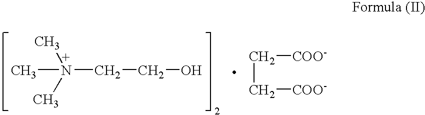

3-5. (canceled)

6. A method for delivering a compound of Formula (I) 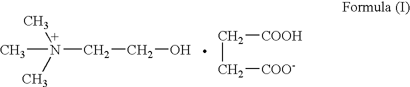
to a mammal in need thereof comprising intranasally administering a pharmaceutical composition comprising a therapeutically effective amount of the compound of Formula (I) or a pharmaceutically acceptable salt thereof, and a pharmaceutically and intranasally acceptable carrier.

7. The method of claim 6, wherein the pharmaceutically acceptable salt is selected from the group consisting of choline salt, sodium salt, potassium salt, and thiamine salt.

8. A method of treating a disease selected from the group consisting of Alzheimer's disease, Parkinson's disease, Huntington's disease, cerebral ischemia and neurological damage due to stroke, diabetic polyneuropathy, and amyotrophic lateral sclerosis; the method comprising intranasally administering to a mammal in need thereof a pharmaceutical composition comprising a therapeutically effective amount of the compound of Formula (I) 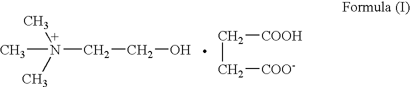
or a pharmaceutically acceptable salt thereof, and a pharmaceutically and intranasally acceptable carrier.

9. The method of claim 8, wherein the pharmaceutically acceptable salt is selected from the group consisting of choline salt, sodium salt, potassium salt, and thiamine salt.
